(19) United States
(12) Patent Application Publication
(10) Pub. No.: US 2017/0290988 A1
(43) Pub. Date: Oct. 12, 2017

(54) An Injection Device Adapted to Detect Air in a Cartridge

(71) Applicant: Novo Nordisk A/S, Bagsvaerd (DK)

(72) Inventors: Michael Monrad, Frederiksberg (DK); Jens Aage Munk, Oelstykke (DK); Per Einar Pontus Holm, Tygelsjoe (SE); Dan Noertoft Soerensen, Alleroed (DK)

(21) Appl. No.: 15/507,861

(22) PCT Filed: Sep. 1, 2015
(86) PCT No.: PCT/EP2015/069889

§ 371 Date: Mar. 1, 2017

(30) Foreign Application Priority Data
Sep. 5, 2014 (EP) 14183758.3

(51) Int. Cl. A61M 5/315; A61M 5/24; A61M 5/31; A61M 5/20

(52) U.S. Cl. CPC A61M 5/31565; A61M 5/20; A61M 5/24; A61M 5/31; A61M 2005/31588; A61M 2205/33; A61M 5/14

(57) ABSTRACT

A drug delivery device (100) for estimating the amount of free air in a cartridge (1) comprising a cartridge receiving portion (not shown) adapted to receive a cartridge (1), the cartridge (1) comprising a body portion (6), an axially displaceable piston (5), and a distal outlet portion (4), an expelling assembly comprising an axially displaceable piston drive member (10) adapted to interface with the proximal piston (5) portion of a received cartridge (1), the piston drive member (10) is adapted to be moveable in a distal direction, and to thereby pressurize the drug in a received cartridge. The drug delivery device further comprises means for measuring and acquiring a property relating to a fluid pres-sure of the drug in a received cartridge (1), and where the property depends on the amount of free air (7) contained in the cartridge (1). The drug delivery device further comprises means (20) for measuring and acquiring the position of the piston rod drive member (10), and means (40) for measuring and acquiring the time. The drug delivery device further comprises means (30) for storing, and means (31) for processing the acquired measurements. The processing means are configured for estimating the amount of air (7) in the cartridge (1).

The present invention generally relates to medical delivery devices. In specific embodiments the invention relates to medical delivery devices adapted for detecting the amount of free air in a cartridge.

### BACKGROUND OF THE INVENTION

In the disclosure of the present invention reference is mostly made to the treatment of diabetes by delivery of insulin, however, this is only an exemplary use of the present invention.

Drug delivery devices have greatly improved the lives of patients who must self-administer drugs and biological agents. Drug delivery devices may take many forms, including simple disposable devices that are little more than an ampoule with an injection means, they may be prefilled disposable devices, or durable devices adapted to be used with pre-filled cartridges. The devises may be connected with different types of needles or infusion sets depending on their application. Regardless of their form and type, they have proven to be great aids in assisting patients to self-administer injectable drugs and biological agents. They also greatly assist care givers in administering injectable medicines to those incapable of performing self-injections.

Users of some existing diabetes injection devices must ensure that there is no air in the cartridge before injections. A new cartridge can be controlled for the amount of free air before it is leaving production. However, when the cartridge is in-use it is possible for air to enter the cartridge if the cartridge is placed in an environment with varying temperature. One such scenario could be a user placing an injection device in a refrigerator for storing, and taking it out for injections. Once the air is in the cartridge, and if the user is not removing it before injection, the user may experience an under dose, as the device may expel air instead of a drug, or because the dosage time is increased with the amount of air in the cartridge, i.e., the user may stop the injection before the intended dose is expelled.

U.S. Pat. No. 4,255,088 describes a liquid pumping system wherein gas within the pump may be detected to indicate an error in the desired flow rate, an alarm can be given if an excessive amount of gas is detected, and the flow rate can be corrected in order to obtain the desired flow rate. The document describes a reciprocating type of pump, which continuously works in pump cycles of suction and compression, and the direction of the fluid flow is regulated by inlet and outlet valves. The document describes a reciprocating pump where the pump during a compression phase can be operated in a sealed state, where inlet and outlet valves are closed. In the sealed state, a signal proportional to the piston position is measured along with pressure measurements from a pressure transducer within the pump chamber. The measurements in the sealed state can be used to estimate the amount of gas in the chamber.

WO 2013/148798 A1 describes different embodiments for detecting air in the pump chamber of an infusion system. The infusion system comprises a reciprocating type of pump and valves to ensure a unidirectional flow. For one embodiment, the document discloses a method for detecting air in a chamber of an infusion system. In one step, a plunger is moved against a chamber containing fluid using an actuator device. In another step, a force acting on the plunger, as it moves against the chamber, is detected with a sensor. In yet another step, a measurement of the force acting on the plunger is electronically communicated from the sensor to a processor. In another step, the processor is used to: (1) preprocess a force profile detected by the sensor (2) extract features from the force profile, and (3) classify the force profile as being an air force profile or a liquid force profile based on the extracted features of the force profile. The used sensor (110) is positioned within the chamber (108) and detects the force acting on the plunger (107), as it moves. The methods are aiming at detecting an end of bag condition, and are therefore adapted to analyse a series of pump cycles.

It is an object of the present invention to provide a drug delivery device which is capable of estimating the amount of air in the cartridge in order to ensure compliance to a prescribed treatment and avoid under dosing.

### DISCLOSURE OF THE INVENTION

In the disclosure of the present invention, embodiments and aspects will be described which will address the above mentioned objects or which will address objects apparent from the below disclosure as well as from the description of exemplary embodiments.

In accordance with a first aspect, the invention provides a drug delivery device for estimating the amount of free air in a cartridge comprising:
a cartridge receiving portion adapted to receive a cartridge, wherein the cartridge comprises a body portion, an axially displaceable piston, and a distal outlet portion,
an expelling assembly comprising an axially displaceable piston drive member adapted to be brougth into an interfacing position in abutment with the proximal piston portion of a received cartridge, the piston drive member is adapted to be moveable in a distal direction, and to thereby pressurize the drug in the received cartridge,
means for measuring and acquiring a property relating to a fluid pressure of the drug in the received cartridge during operation, and where the property depends on the amount of free air contained in the cartridge,
means for measuring and acquiring the position of the piston drive member during operation,
means for storing the acquired measurements during operation, and
processing means for processing the acquired measurements during operation, and for estimating the amount of free air in the cartridge.

By this arrangement of the drug delivery device, it is possible to estimate the amount of air in a cartridge, in a system, where the cartridge is received in a cartridge receiving portion. The drug contained in the cartridge is pressurized by a piston, and the piston rod member is adapted to be interfaced with a piston drive member. The piston drive member is moving distally in order to pressurize the drug. As air is compressible and drug is very little compressible it is possible to determine the amount of air from the correlation between piston position and a response in the property related to the fluid pressure. A drug delivery device of the above mentioned type is adapted to receive a cartridge, in a cartridge receiving portion, and to expel the drug. In such systems the piston drive member is adapted to be interfaced with the piston of the cartridge in a slidable manner without interlocking means between the piston drive member and the piston. In other words the piston is not locked to the piston drive member, and this constructional feature imposes limitations on how the processing means can estimate the amount of free air in the cartridge. When the cartridge is empty, a new standard cartridge may be inserted or the device may be discarded if it is of the prefilled type.

Also, in a further aspect of the invention, the piston drive member (10) and the piston (5) are adapted to be changeable between a non-contacting configuration, wherein the piston drive member (10) is positioned with an axial distance relative to the piston (5), and a contacting configuration, wherein the piston drive member (10) is interfacing and abutting the piston (5), wherein further movement of the piston drive member (10) in the distal direction, will result in a distal movement of the piston (5), and increase the fluid pressure of the drug in the received cartridge (1), and wherein a movement of the piston drive member (10) in a proximal direction results in a separation between the piston drive member (10) and the piston (5). Thereby, the pressure in the cartridge will decrease.

The piston drive member is adapted to be in a non-contact configuration, where the drive member is axially separated from the piston, i.e., there is an axial distance between the drive member and the piston, or in a contact configuration wherein there is an abutment between the piston and the piston drive member, but where the piston drive member and the piston can be separated, if the piston drive member is moved in the proximal direction.

In a further aspect the piston drive member (10) and the piston (5) are adapted to be changed from the non-contacting to the contacting configuration by moving the piston drive member (10) in a distal direction, and the piston drive member (10) and the piston (5) are adapted to be changed from the contacting to the non-contacting configuration by moving the piston drive member (10) in the proximal direction.

By this arrangement it is possible to change the configuration of the piston drive member and the piston by moving the piston drive member in a distal or proximal direction. As the piston drive member, in the contact configuration, only is in abutment with the piston, a proximal movement of the piston drive member can create an axial distance to the piston.

The property related to the fluid pressure may be obtained by a force sensor placed between the piston and the piston drive member, in this case the property related to the fluid pressure is the force exerted on the piston. Also or alternatively, the property related to the fluid pressure may be obtained by a proximity sensor adapted to measure the deformation of the piston. When the piston is exerted to a force from the piston drive member, it will deform, and the deformation is related to the fluid pressure inside the cartridge. Also or alternative the deformation of the piston or the plunger could be measured by a strain gauge.

Also or alternatively, the property related to the fluid pressure may be obtained by measuring the capacitance or the absorbance of the fluid within the cartridge. The capacitance and absorbance of gas or air is different from liquid and drug. When the piston is pressurized the ratio between gas and liquid within the cartridge will change, and by measuring capacitance or absorbance of the fluid within the cartridge, the fluid pressure can be derived.

In a further aspect, the invention provides a drug delivery device where the outlet portion is adapted to be operated in a first mode, where the distal outlet is closed.

By this arrangement it is possible to enable a particular simple method for determining the amount of air, as there will be a linear relationship between the fluid pressure and position of the piston drive member, with the assumption that the temperature is constant. The distal outlet is for example closed when it is not connected with a flow conduit.

In a further aspect the invention provides a drug delivery device where processing means are adapted to estimate the contact point between the piston drive member and the piston, by using the measured property relating to the fluid pressure.

By this arrangement it is possible to detect that there is contact between the piston and the piston drive member, and thereby ensure that there will be an increase in pressure when the piston drive member is moved further.

In a further aspect the invention provides a drug delivery device where the processing means are further configured for processing the acquired measurements and eliminate the effect of a distance or slack between piston rod member and the piston.

By this arrangement it is not necessary to positively determine the contact point, as the processor is configured for eliminating the effect of a distance between the piston drive member and the piston during the estimation of free air in the cartridge.

In a further aspect the invention provides a drug delivery device, where the means for measuring the property relating to the fluid pressure is mounted on a distal portion of the piston drive member. In a further aspect this arrangement, the means for measuring said property could be a force sensor or a proximity sensor mounted in a sensor head at the distal portion of the piston drive member. In yet a further aspect the force sensor and the proximity sensor may be mounted in a combined arrangement in the sensor head.

By this arrangement the means for measuring the property relating to the fluid pressure is not mounted on the cartridge. Therefore, this arrangement enables a method where the cartridge can be replaced, and it further allows for the use of a standard cartridge.

In a further aspect the invention provides a drug delivery device further comprising
means for measuring and acquiring the time during operation,
where the outlet portion further is adapted to be arranged in a second mode, where the distal outlet is in fluid communication with one of different flow conduits, and
where the piston drive member is adapted to be moveable in a distal direction to expel a selected amount of drug, and
where the processing means are further configured for estimating at least the amount of free air in the cartridge during operation.

By this arrangement it will be possible to enable a method of estimating the amount of air in the cartridge, in a mode where the drug is expelled, as there is a time dependent correlation between the fluid pressure and the position of the piston drive member. The measured time is a time stamp identifying the time of all events. The fluid pressure is further depended on the selected amount of drug to be delivered, dose delivery speed, the amount of free air in the cartridge, different amounts of friction between the piston and the body portion of the cartridge, different type of flow conduits etc.

In a further aspect the invention provides a drug delivery device further comprising means for identifying whether the outlet portion is arranged in the first or the second mode, and where the processing means are configured for using the identification in estimating at least the amount of free air in the cartridge.

By this arrangement it is possible to simplify the method of estimating the amount of air, as the processing means are provided with information of the operation mode. The means for identifying the operation mode, can for example be the user visually inspecting the outlet portion and providing the information to the processor, or it can be a build in sensor identifying that the outlet portion has been connected with a flow conduit, and the processor is provided with the information automatically.

In a further aspect the invention provides a drug delivery device, where the expelling assembly has been adapted to be operated in a stroke phase, wherein the piston is adapted to be moved in a distal direction by the piston drive member, and in a relaxation phase, wherein the piston drive member is adapted to be stopped, i.e., the drive member is not driven and does not move. The processing means is further configured for estimating the amount of free air based on data acquired during the relaxation phase.

By this arrangement the amount of drug will be delivered during a stroke phase, where the piston drive member is advanced, and where the compliant parts, like free air, are compressed, and a relaxation phase, where all the compliant parts relaxes. It is a particular advantage to be able to acquire and process measurements during the relaxation phase, as the piston is stopped. Estimation of the amount of free air based on data acquired during the relaxation phase is therefore less influenced by effect of friction between the piston and the body portion of the cartridge. Friction or, more specific, dynamic friction does only occur when the piston is advancing.

In a further aspect the invention provides a drug delivery device where the processing means are configured for
processing the acquired measurements, and
performing a running estimate of at least the amount of free air in the cartridge.

This arrangement enables a method where the estimate of the amount of free air is continuously improved along with the increased acquisition of measurements. Estimates of the amount of free air during the relaxation phase are particular suitable for a qualified estimate, as the contribution from friction has been minimized.

In a further aspect the invention provides a drug delivery device where
the means for storing are provided with a set of calibration indicators calculated from calibration measurements, where
the processing means are configured for
   calculating a set of operation indicators, and
   using the set of operation indicators and the set of calibration indicators to estimate at least the amount of free air in the cartridge during operation.

By this arrangement a set of calibration indicators, stored on the storing means, enables a method of estimating several unknown parameters during operation, by extracting a set of operation indicators and comparing said set with the set of calibration indicators. The storing means are provided with the set of calibration indicators before, the drug delivery device is operational, i.e., before it can be used to deliver a dose to a patient, and estimate the amount of free air. The set of calibration indicators represents measurements on cartridges with different amounts of free air, performed with different selected amounts of drug to be delivered, different amounts of friction between piston and body portion of cartridge, different velocities of the piston etc.

In a further aspect, the invention provides a drug delivery device, where the processing means are configured for at least estimating the amount of free air in the cartridge based on a physical model.

This arrangement enables a method, where it is possible to estimate at least the amount of free air in the cartridge, by the establishment of a physical model linking time during operation, the amount of free air in the cartridge, the amount of delivered drug, friction between piston and body portion of cartridge, velocity of the piston etc.

In a further aspect, the drug delivery device further comprises a cartridge.
In a further aspect, the invention provides a method for operating a drug delivery device according to the invention comprising the steps of:operating the expelling means to axially displace the piston,
measuring and acquiring the property relating to the fluid pressure of the drug during operation,
measuring and acquiring the position of the piston drive member during operation,
processing the acquired measurements to estimate at least the amount of free air in the cartridge.

In a further aspect, the invention further comprises the step of changing the configuration of the piston drive member and the piston from the non-contacting configuration to the contacting configuration,

In a further aspect, the invention provides a method for operating the drug delivery device according to the invention further comprising the steps of:
identifying whether the outlet portion is in the first mode or the second mode, and
using said identification as an input to the estimation of the calculated volume of free air in the cartridge.

In a further aspect, the invention provides a method of operating a drug delivery device according to the invention further comprising the steps of:
measuring and acquiring the time during operation,
providing a set of calibration indicators calculated from calibration measurements,
calculating a set of operation indicators, and
using the set of operation indicators and the set of calibration indicators to estimate at least the amount of free air in the cartridge during operation.

In a further aspect, the invention provides a method for operating a drug delivery device according to the invention further comprising the steps of:
measuring and acquiring the time during operation, and
estimating at least the amount of free air in the cartridge based on a physical model.

In a further aspect, the invention provides a method for operating a drug delivery device according to the invention further comprising producing an alert if the estimated amount of free air in the cartridge is above a certain threshold.

In a further aspect, the invention provides a method for operating a drug delivery device according to the invention further comprising the steps of:
measuring and acquiring a property relating to the fluid pressure of the drug in the received cartridge in the stroke phase and the relaxation phase,
measuring and acquiring the position of the piston drive member in the stroke phase and the relaxation phase,
measuring and acquiring the time in the stroke phase and the relaxation phase, and
processing the acquired measurements to estimate at least the amount of free air in the cartridge.

### BRIEF DESCRIPTION OF THE DRAWINGS

In the following the invention will be further described with reference to the drawings, wherein

FIG. 1 shows schematically components of a drug delivery device according to the invention

FIG. 2a shows schematically a force measurement when an outlet portion of the drug delivery device is in the first closed mode

FIG. 2b shows schematically a force measurement when the outlet portion of the drug delivery device is in second connected mode

FIG. 3a shows schematically the intensity signal of a proximity sensor head as it approaches and pressurizes a piston and the fluid contained in the cartridge.

FIG. 3b shows schematically the relation between the intensity signal of the proximity sensor and the fluid pressure, as the proximity sensor head pressurizes the fluid contained in the cartridge.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not to an actual situation of use. In the following the term proximal is used to indicate the end or portion closest to a user administrating the drug delivery device, and the term distal is used to refer to a portion or end opposite to the proximal portion or end. The shown figures are schematic representations for which reason the configuration of the different structures as well as the relative dimensions are intended to serve illustrative purposes only.

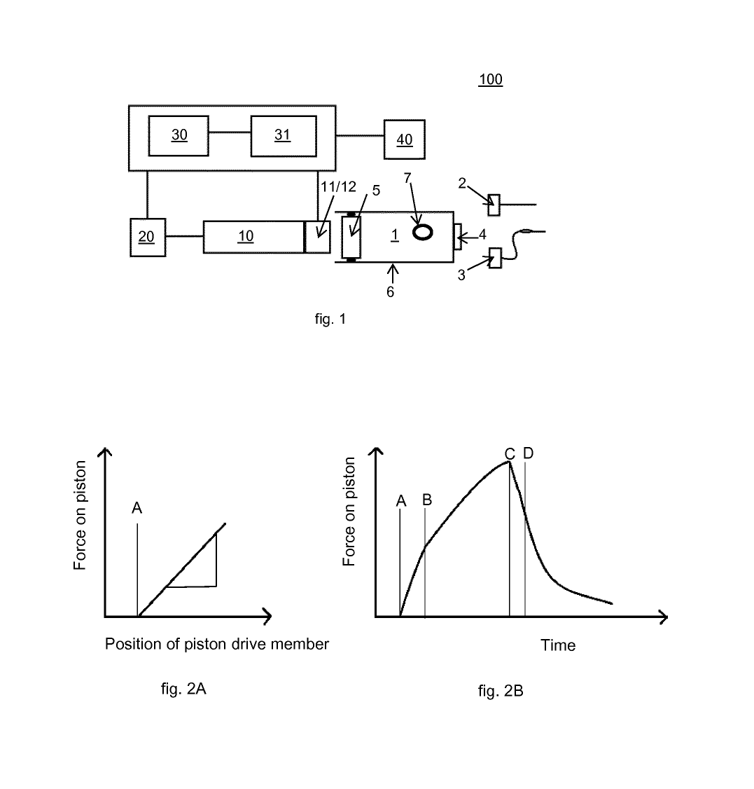

In a first embodiment of the disclosure, the drug delivery device (100) of FIG. 1 may determine the amount of air in the cartridge. FIG. 1 shows the drug delivery device (100) comprising

a cartridge receiving portion (not shown) adapted to receive a cartridge (1), the cartridge (1) comprising a body portion (6), an axially displaceable piston (5), and a distal outlet portion (4), an expelling assembly comprising an axially displaceable piston drive member (10) adapted to interface with the proximal piston (5) portion of a received cartridge (1), the piston drive member (10) is adapted to be moveable in a distal direction, and to thereby pressurize the drug in a received cartridge

The drug delivery device further comprises means for measuring and acquiring a property relating to a fluid pressure of the drug in a received cartridge (1), and where the property depends on the amount of free air (7) contained in the cartridge (1), where the means for measuring this property in a preferred embodiment is a force sensor (11), a proximity sensor (12) or a combination of both sensors mounted in a sensor head, at the end of the piston drive member (10).

The property related to the fluid pressure may be obtained by a force sensor placed between the piston and the piston drive member, in this case the property related to the fluid pressure is the force exerted on the piston. Also or alternatively, the property related to the fluid pressure may be obtained by a proximity sensor adapted to measure the deformation of the piston. When the piston is exerted to a force from the piston drive member, it will deform, and the deformation is related to the fluid pressure inside the cartridge. Also or alternative the deformation of the piston or the plunger could be measured by a strain gauge.

In an alternative embodiment the property related to the fluid pressure may be obtained by measuring the capacitance or the absorbance within the cartridge. The capacitance and absorbance of gas or air is different from liquid and drug. When the piston is pressurized the ratio between gas and liquid within the cartridge will change, and by measuring capacitance or absorbance within the cartridge, the fluid pressure can be derived. In such an embodiment means for measuring the capacity or the absorbance are preferably provided along an axial extension of the cartridge.

The drug delivery device further comprises means (20) for measuring and acquiring the position of the piston drive member (10) during operation, which in a preferred embodiment is an encoder, or a position sensor using electric contact or magnetic signals.

The drug delivery device further comprises means for storing (30) the acquired measurements, and processing means (31) for processing the acquired measurements during operation, and for estimating the amount of free air in the cartridge (1). The piston drive member (10) and the piston (5) are adapted to be changeable between a non-contacting configuration, wherein the piston drive member (10) is positioned with an axial distance relative to the piston (5), and a contacting configuration, wherein the piston drive member (10) is interfacing and is in abutment with the piston (5). Furthermore movement of the piston drive member (10) in the distal direction will result in a distal movement of the piston (5), and the movement will result in an increase of the fluid pressure of the drug in the received cartridge (6). A movement of the piston drive member (10) in a proximal direction will allow the piston to move in a proximal direction, which will result in a separation between the pisto drive member (10) and the piston, and in a decrease in the fluid pressure. The distal direction is in the direction of the outlet, and the proximal direction is the direction opposite to the distal direction.

The drug delivery device can further comprise means for measuring and acquiring the time (40) during operation, in order to be able to determine the amount of free air under conditions, where the outlet portion (4) is connected to a flow conduit, and the fluid pressure therefore will be dependent on time.

In order to store the acquired data, the drug delivery device is provided with means for storing the acquired measurements (30) during operation, and processing means (31) for processing the acquired measurements during operation, and for estimating the amount of free air (7) in the cartridge (1).

The piston drive member (10) is moving distally in order to pressurize the drug. As air is compressible and drug is very little compressible it is possible to determine the amount of free air (7) from the correlation between piston position and a response in the property related to the fluid pressure within the cartridge.

In a particular simple embodiment the invention discloses a drug delivery device where the outlet portion (4) is adapted to be operated in a first mode, where the distal outlet is closed. In a more advanced embodiment, the drug delivery device can be operated in a second mode where the outlet portion (4) is connected with the flow conduit as a pen needle (2) or an infusion set (3). Instead of an embodiment where the drug delivery device is adapted to operate in one of the two modes, an embodiment where a user selects between the two modes depending on an observation of the outlet portion (4), or an embodiment where the drug delivery device automatically identifies the operation mode, depending on the state of the outlet portion, i.e, a sensor to detect connectivity with a flow conduit could be associated with the outlet portion.

In the first mode, it is possible to enable a particular simple method for determining the amount of air. FIG. 2A illustrates schematically the output of a measurement with a force sensor (12), as a function of the position of the piston drive member, when the outlet portion (4) is in the first mode. The point A indicates contact between the piston drive member (10) and the piston (5), and after this point there will be a linear relationship between the fluid pressure and the position of the piston drive member, with the assumption that the temperature is constant. After the point A, any further advancement of the piston drive member (10) will create a response in the fluid pressure. As the pressure and the position of the point A, and the pressure and the position after a further advancement of the piston drive member (10) can be obtained, it is possible to determine the amount or volume of air (7) at the point A, when no pressure is applied. The volume at the point A is considered as the initial volume, and if the initial volume is larger than a defined critical amount, the user can be alarmed.

FIG. 2B schematically illustrates the output of a measurement with a force sensor (12) as a function of time, when the output portion (4) is in the second mode. The point A indicates contact between the piston drive member (10) and the piston (5), the point A also indicates the start of an acceleration phase, where the piston drive member (10) accelerates the piston (5), and pressurizes and expels the fluid. The point B indicates the end of the acceleration phase, and the beginning of a phase, where the piston drive member (10) moves with a constant speed. The point C indicates the end of the constant speed phase, and the beginning of a deceleration phase, where the fluid is still expelled although the fluid pressure decreases. The point D indicates the end of the deceleration phase, and the beginning of a relaxation phase, where the piston drive member has stopped, but where drug is still expelled to the relaxation of compliant parts as for example free air. If air is present in the cartridge it will usually be the main contributing part to the overall compliance of the system, but other components like piston, piston drive member, and septum may also contribute. The relaxation phase ends, when the fluid pressure is equal to the ambient pressure. At this point, no more drug is expelled. The acceleration phase, the constant speed phase, and the deceleration phase are in common referred to as the stroke phase, as the piston drive member is advancing in all phases.

For this type of drug delivery device, a selected amount of drug will be expelled during a stroke phase and a relaxation phase, which is contrary to continuously reciprocating systems, where the cartridge or pump chamber will not release pressure during a relaxation phase, as the piston is reversed with the piston drive member, before the compliant parts have fully relaxed.

The sensor measuring the force between a piston drive member (10) and a piston (5), with a constant cross sectional area is easily related to the fluid pressure inside the cartridge (1). The general relation is that pressure equals force per unit area.

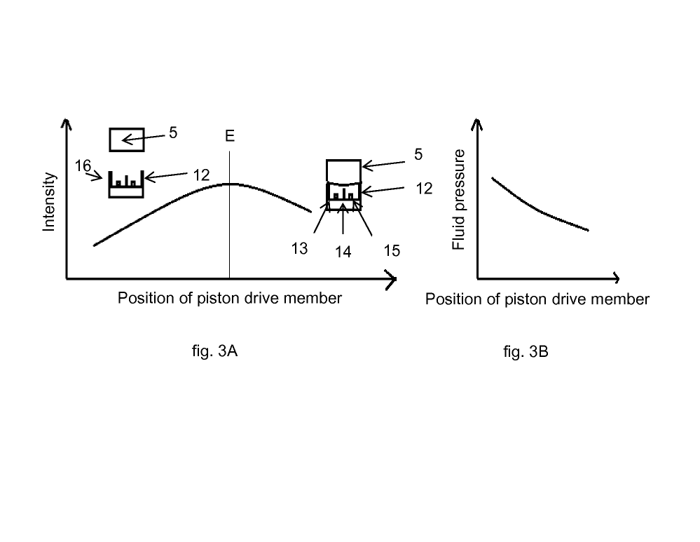

The proximity sensor can be used to detect plunger contact, as disclosed in WO 2013/144152, and hereby incorporated by reference. As schematically illustrated in FIGS. 3A and 3B the intensity signal from the proximity sensor can also be related to the fluid pressure of the fluid in the cartridge (10). FIG. 3A illustrates the output of a measurement with a proximity sensor (12) mounted in a sensor head (16), on a distal portion of the piston drive member (10), as the sensor head (16) approaches the piston (5). An emitter (13) sends a signal which is reflected by a proximal surface of the piston (5), and detected by a detector (15). A separating wall (14) shields the detector (15) towards direct signals from the emitter (13). As the sensor head (16) approaches the piston (5) the intensity of the reflected signal increases until contact between a distal surface of the sensor head (16) and the proximal surface of the piston (5). After contact and in the case of further advancement of the piston drive member (10), the piston (5) pressurizes the fluid in the cartridge and expands into a distal cavity of the sensor head (16), whereby the intensity of the signal decreases. In this phase the correlation between the intensity and the fluid pressure can be obtained, and the correlation is schematically illustrated in FIG. 3B.

FIGS. 2 and 3 illustrates that a drug delivery device according to this invention comprising means for measuring the property relating to the fluid pressure, e.g., force or proximity signal, also can be used to estimate the contact point between piston rod member (10) and piston (5). In the case of further advancement of the piston drive member, after contact, the pressure will further increase.

However, an embodiment of the present invention is not necessarily adapted to determine the contact point in a separate or initial step, as it is also possible to configure the processor, by applying a suitable algorithm, to eliminate the effect of a distance or slack between piston drive member and piston, during the estimation of the amount of free air in the cartridge.

Another advantage of an embodiment according to the present invention is that when the sensing means are mounted on the piston drive member, the cartridge can easily be replaced without interfering with the sensor, or it will be possible to use a drug delivery device with a standard cartridge, which is reducing the production costs.

In an embodiment where the drug delivery device is to be operated in the second mode, the drug delivery device, as previously explained, also comprises means for measuring and acquiring the time during operation.

In a preferred embodiment of the invention the processing means of the drug delivery device are configured for instantly processing the acquired measurements, and performing a running estimate of at least the amount of free air in the cartridge. This arrangement enables a method where the estimate of the amount of free air is continuously improved along with the increased acquisition of measurements.

In a preferred embodiment of the invention the means for storing (30) are provided with a set of calibration indicators calculated from calibration measurements, where the processing means (31) are configured for calculating a set of operation indicators, and using the set of operation indicators, and the set of calibration indicators to estimate at least the amount of free air in the cartridge during operation.

When using the drug delivery device several parameters can be varied by the user like the selected amounts of drug to be delivered and dose delivery speed. Information of the selected parameters can be provided to the processor prior to dosing. Other variables like the amount of free air in the cartridge, different amounts of friction between piston and body portion of the cartridge, different type of flow conduits (different amount of flow resistance) are typically unknown variables prior to dosing.

Therefore, the set of calibration indicators are calculated based on measurements of a property relating to the fluid pressure, for various selected amounts of drug to be delivered and obtionally also one or more from the following group of parameters: dose delivery speed, different types of flow conduit, the amount of free air in the cartridge, different amounts of friction between piston and body portion of the cartridge. The set of calibration indicators are indicators used to characterize special features for the variation of the fluid pressure during the delivery of a dose for all possible conditions.

When a user starts a delivery of a selected amount of drug, and when the drug is in the second mode, the processing means (31) starts to process the measured and acquired measurements (a property relating to the fluid pressure, position of the piston drive member, time during operation), and applies an algorithm to calculate a new set of indicators, operation indicators, which comprises indicators used to characterize the fluid pressure during the current delivery. The set of operation indicators is continuously calculated, and compared with the set of calibration indicators in order to at least estimate the amount of free air in the cartridge, and optionally also one or more of the following group of parameters: different types of flow conduit, different amounts of friction between piston and body portion of the cartridge

By this arrangement a set of calibration indicators, stored on the storing means, enables a method of characterizing the response of the fluid pressure during delivery, and for estimating several unknown parameters during operation, by extracting a set of operation indicators and comparing the set with the set of calibration indicators. The storing means are provided with the set of calibration indicators before the drug delivery device can be operational in the sense that it can be used for delivering an amount of drug to a patient. The set of calibration indicators can for example be provided before the drug delivery device is leaving production.

In another preferred embodiment the processing means are configured for at least estimating the amount of free air in the cartridge based on a physical model.

This arrangement enables a method, where it is possible to estimate at least the amount of free air in the cartridge, by the establishment of a physical model linking time during opera-tion, the amount of free air in the cartridge, the amount of selected drug to be, friction between piston and body portion of cartridge, dose delivery speed etc.

When estimating the amount of air in the cartridge in the second mode, it can in particular be useful to process data from the relaxation phase, as the plunger in this phase has stopped movement, and there is consequently a minimal influence from the dynamic friction between the piston and the body portion of the cartridge.

In an exemplary method of using the embodiments according to the invention, the method comprises the steps of operating the expelling means to axially displace the piston, measuring and acquiring the property relating to the fluid pressure of the drug during operation, measuring and acquiring the position of the piston drive member during operation, processing the acquired measurements to estimate at least the amount of free air in the cartridge.

In an exemplary method of using the embodiments according to the invention, the method comprises the steps of identifying whether the outlet portion is in the first mode or the second mode, and using said identification as an input to the estimation of the calculated volume of free air in the cartridge.

In an exemplary method of using the embodiments according to the invention, the method comprises the steps of measuring and acquiring the time during operation, providing a set of calibration indicators calculated from calibration measurements calculating a set of operation indicators, and using the set of operation indicators and the set of calibration indicators to estimate at least the amount of free air in the cartridge during operation.

In an exemplary method of using the embodiments according to the invention, the method comprises the steps of measuring and acquiring the time during operation, and estimating at least the amount of free air in the cartridge based on a physical model.

In an exemplary method for operating a drug delivery device according to the invention, the method further comprising producing an alert if the estimated amount of free air in the cartridge is above a certain threshold.

In an exemplary method for operating a drug delivery device according to the invention, the method further comprises the steps of: measuring and acquiring a property relating to the fluid pressure of the drug in the received cartridge in the stroke phase and the relaxation phase, measuring and acquiring the position of the piston drive member in the stroke phase and the relaxation phase, measuring and acquiring the time in the stroke phase and the relaxation phase, and processing the acquired measurements to estimate at least the amount of free air in the cartridge.

### List of Embodiments

1. A drug delivery device (100) for estimating the amount of free air in a cartridge comprising:
   a cartridge receiving portion adapted to receive a cartridge (1), wherein the cartridge (1) comprises a body portion (6), an axially displaceable piston (5), and a distal outlet portion (4),
   an expelling assembly comprising an axially displaceable piston drive member (10) adapted to interface with the proximal piston (5) portion of a received cartridge, the piston drive member (10) is adapted to be moveable in a distal direction, and to thereby pressurize the drug in the received cartridge (1),
   means (11/12) for measuring and acquiring a property relating to a fluid pressure of the drug in the received cartridge (1), and wherein the property depends on the amount of free air (7) contained in the cartridge,
   means (20) for measuring and acquiring the position of the piston drive member (10),
   means for storing (30) the acquired measurements, and
   processing means (31) configured for processing the acquired measurements, and for estimating the amount of free air (7) in the cartridge (1).
2. A drug delivery device according to embodiment 1, wherein the piston drive member (10) and the piston (5) are adapted to be changeable between a non-contacting configuration, wherein the piston drive member (10) is positioned with an axial distance relative to the piston (5), and a contacting configuration, wherein the piston drive member (10) is interfacing and abutting the piston (5), wherein further movement of the piston drive member (10) in the distal direction, will result in a distal movement of the piston (5), and increase the fluid pressure of the drug in the received cartridge (1), and wherein a movement of the piston drive member (10) in a proximal direction results in a separation between the piston drive member (10) and the piston (5).
3. A drug delivery device according to any of the previous embodiments, wherein the piston drive member (10) and the piston (5) are adapted to be changed from the non-contacting to the contacting configuration by moving the piston drive member (10) in a distal direction, and wherein the piston drive member (10) and the piston (5) are adapted to be changed from the contacting to the non-contacting configuration by moving the piston drive member (10) in the proximal direction.
4. A drug delivery device according to any of the embodiments 1-2, wherein the outlet portion (4) is adapted to be operated in a first mode, where the distal outlet is closed.
5. A drug delivery device according to any of the previous embodiments, wherein the processing means (31) are further configured for processing the acquired measurements and eliminate the effect of a distance between the piston drive member (10) and the piston (5).
6. A drug delivery device according to any of the previous embodiments, wherein the means (11, 12) for measuring the property relating to the fluid pressure, is mounted on a distal portion of the piston drive member (10).
7. A drug delivery device according to any of the previous embodiments, further comprising
   means (40) for measuring and acquiring the time,
   where the outlet portion (4) further is adapted to be arranged in a second mode, where the distal outlet portion (4) is in fluid communication with one of different flow conduits (2, 3), and
   where the piston drive member (10) is further adapted to expel a selected amount of drug.
8. A drug delivery device according to embodiment 7 further comprising means for identifying whether the outlet portion (4) is arranged in the first or the second mode, and where the processing means (31) are configured for using the identification in estimating at least the amount of free air (7) in the cartridge (1).
9. A drug delivery device according to any of the embodiments 7-8, where the expelling assembly has been adapted to be operated in a stroke phase, wherein the piston is moved in a distal direction by the piston drive member, and in a relaxation phase, wherein the piston drive member is stopped, and wherein the processing means (31) is further configured for estimating the amount of free air based on data acquired during the relaxation phase.
10. A drug delivery device according to any of the previous embodiments, wherein the processing means (31) are further configured for performing a running estimate of at least the amount of free air (7) in the cartridge.
11. A drug delivery device according to any of the embodiments 7-10, wherein
   the means for storing are provided with a set of calibration indicators calculated from calibration measurements, where
   the processing means (31) are further configured for
      calculating a set of operation indicators, and
      using the set of operation indicators and the set of calibration indicators to estimate at least the amount of free air (7) in the cartridge (1) during operation.
12. A drug delivery device according to any of the embodiments 7-10, where the processing means (31) are configured for at least estimating the amount of free air (7) in the cartridge (1) based on a physical model.
13. A method for operating a drug delivery device according to any of the embodiments 1-12 comprising the steps of:
   changing the configuration of the piston drive member (10) and the piston (5) from the non-contacting configuration to the contacting configuration,
   operating the expelling means to axially displace the piston (5),
   measuring and acquiring the property relating to the fluid pressure of the drug,
   measuring and acquiring the position of the piston drive member during operation,
   processing the acquired measurements to estimate at least the amount of free air in the cartridge (1).
14. A method according to embodiment 13 for operating the drug delivery device according to any of the embodiments—7-12 further comprising the steps of:
   identifying whether the outlet portion (4) is in the first mode or the second mode, and
   using said identification as an input to the estimation of the calculated volume of free air (7) in the cartridge.
15. A method according to any of the embodiments 13-14 for operating a drug delivery device according to any of the embodiments 7-12 further comprising the steps of:
   measuring and acquiring the time,
   providing a set of calibration indicators calculated from calibration measurements,
   calculating a set of operation indicators, and
   using the set of operation indicators and the set of calibration indicators to estimate at least the amount of free air in the cartridge (1) during operation.
16. A method according to any of the embodiments 13-15 for operating a drug delivery device according to any of the embodiments 7-12 further comprising the step of producing an alert if the estimated amount of free air in the cartridge (1) is above a certain threshold.
17. A method according to embodiments 13-16 for operating a drug delivery device according to embodiments 5-10 further comprising the steps of:
   measuring and acquiring a property relating to the fluid pressure of the drug in the received cartridge (1) in the stroke phase and the relaxation phase,
   measuring and acquiring the position of the piston drive member (10) in the stroke phase and the relaxation phase,
   measuring and acquiring the time in the stroke phase and the relaxation phase, and processing the acquired measurements to estimate at least the amount of free air (7) in the cartridge (1).

1. A drug delivery device for estimating the amount of free air in a cartridge comprising:
a cartridge receiving portion adapted to receive a cartridge, wherein the cartridge comprises a body portion, an axially displaceable piston, and a distal outlet portion,
an expelling assembly comprising an axially displaceable piston drive member adapted to interface with the proximal piston portion of a received cartridge, the piston drive member is adapted to be moveable in a distal direction, and to thereby pressurize the drug in the received cartridge,
a structure for measuring and acquiring a property relating to a fluid pressure of the drug in the received cartridge, and wherein the property depends on the amount of free air contained in the cartridge,
a structure for measuring and acquiring the position of the piston drive member,
a structure for storing the acquired measurements, and
a processing structure configured for processing the acquired measurements, and for estimating the amount of free air in the cartridge.

2. A drug delivery device according to claim 1, wherein the piston drive member and the piston are adapted to be changeable between a non-contacting configuration, wherein the piston drive member is positioned with an axial distance relative to the piston, and a contacting configuration, wherein the piston drive member is interfacing and abutting the piston, wherein further movement of the piston drive member in the distal direction, will result in a distal movement of the piston, and increase the fluid pressure of the drug in the received cartridge, and wherein a movement of the piston drive member in a proximal direction results in a separation between the piston drive member and the piston.

3. A drug delivery device according to claim 1, wherein the piston drive member and the piston are adapted to be changed from the non-contacting to the contacting configuration by moving the piston drive member in a distal direction, and wherein the piston drive member and the piston are adapted to be changed from the contacting to the non-contacting configuration by moving the piston drive member in the proximal direction.

4. A drug delivery device according to claim 1, wherein the outlet portion is adapted to be operated in a first mode, where the distal outlet is closed.

5. A drug delivery device according to claim 1, wherein the processing structure is further configured for processing the acquired measurements and eliminate the effect of a distance between the piston drive member and the piston.

6. A drug delivery device according to claim 1, wherein the structure for measuring the property relating to the fluid pressure, is mounted on a distal portion of the piston drive member.

7. A drug delivery device according to claim 1, further comprising
a structure for measuring and acquiring the time,
where the outlet portion further is adapted to be arranged in a second mode, where the distal outlet portion is in fluid communication with one of different flow conduits, and
where the piston drive member is further adapted to expel a selected amount of drug.

8. A drug delivery device according to claim 7 further comprising a structure for identifying whether the outlet portion is arranged in the first or the second mode, and where the processing structure is configured for using the identification in estimating at least the amount of free air in the cartridge.

9. A drug delivery device according to claim 7, where the expelling assembly has been adapted to be operated in a stroke phase, wherein the piston is moved in a distal direction by the piston drive member, and in a relaxation phase, wherein the piston drive member is stopped, and wherein the processing structure is further configured for estimating the amount of free air based on data acquired during the relaxation phase.

10. A drug delivery device according to claim 1, wherein the processing structure is further configured for performing a running estimate of at least the amount of free air in the cartridge.

11. A drug delivery device according to claim 7, wherein
the structure for storing is provided with a set of calibration indicators calculated from calibration measurements, where
the processing structure is further configured for
calculating a set of operation indicators, and
using the set of operation indicators and the set of calibration indicators to estimate at least the amount of free air in the cartridge during operation.

12. A drug delivery device according to claim 7, where the processing structure is configured for at least estimating the amount of free air in the cartridge based on a physical model.

13. A method for operating a drug delivery device according to claim 1, comprising the steps of:
operating the expelling structure to axially displace the piston,
measuring and acquiring the property relating to the fluid pressure of the drug,
measuring and acquiring the position of the piston drive member during operation,
processing the acquired measurements to estimate at least the amount of free air in the cartridge.

14. A method according to claim 13 for operating a drug delivery device further comprising the steps of:
identifying whether the outlet portion is in the first mode or the second mode, and
using said identification as an input to the estimation of the calculated volume of free air in the cartridge.

15. A method according to claim 13 for operating a drug delivery device further comprising the steps of:
measuring and acquiring the time,
providing a set of calibration indicators calculated from calibration measurements,
calculating a set of operation indicators, and
using the set of operation indicators and the set of calibration indicators to estimate at least the amount of free air in the cartridge during operation.
